(19) United States
(12) Patent Application Publication
(10) Pub. No.: US 2011/0263865 A1
(43) Pub. Date: Oct. 27, 2011

(54) POLYMORPHS OF ZOLMITRIPTAN

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Thungathurthy Srinivasa Rao, Hyderabad (IN)

(73) Assignee: HETERO RESEARCH FOUNDATION, Hyderabad, Andhrapradesh (IN)

(21) Appl. No.: 13/120,449

(22) PCT Filed: Dec. 24, 2008
(86) PCT No.: PCT/IN08/00863

§ 371 Date: Mar. 23, 2011

(30) Foreign Application Priority Data
Dec. 24, 2008 (IN) PCT/IN2008/000863

(51) Int. Cl. C07D 413/02

(52) U.S. Cl. 548/229

(57) ABSTRACT

The present invention provides a novel isopropyl acetate solvate form of zolmitriptan, and a process for its preparation thereof. The present invention also provides a process for preparation of zolmitriptan polymorph A. Thus, for example, zolmitriptan isopropanol solvate was dissolved in isopropyl acetate at 25 deg C, the contents were heated to reflux for 30 minutes and the separated solid was filtered, washed with isopropyl acetate to give zolmitriptan isopropyl acetate solvate.

### FIELD OF THE INVENTION

The present invention provides a novel isopropyl acetate solvate form of zolmitriptan, and a process for its preparation thereof. The present invention also provides a process for preparation of zolmitriptan polymorph A.

### BACKGROUND OF THE INVENTION

Zolmitriptan is known by the chemical name (4S)-4-[[3-[2-(dimethylamino)ethyl]-1H-indol-5-yl]methyl]-2-oxazolidinone. Zolmitriptan is represented by the following structure.

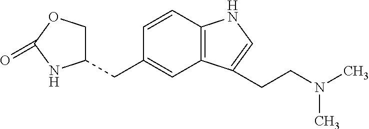

Zolmitriptan is a selective 5-hydroxytryptaminel 1B/1D (5-HT_{1B/1D}) receptor agonist and is of particular use in the treatment of migraine and associated conditions. Commercial products comprising zolmitriptan are available under the name ZOMIG™ in the forms of tablets, orally disintegrating tablets, and nasal spray.

Zolmitriptan may be prepared using the procedures described in U.S. Pat. No. 5,466,699.

WO Patent Publication No. 2005/075467 described various crystalline forms of zolmitriptan and gives processes for their preparation. The Publication described the formation of several crystalline forms of zolmitriptan, which were designated zolmitriptan polymorph A, polymorph B (1-butanol solvate), polymorph C (anisole solvate), polymorph D (isopropanol solvate), polymorph E (ethyl methyl ketone solvate), polymorph F (tetrahydrofuran solvate), polymorph G (1,4-dioxane solvate) and also disclosed is an amorphous zolmitriptan, obtained as an oil from evaporation of a solution of zolmitriptan. WO Patent Publication No. 2005/075467 reported that polymorph A may be prepared by suspending zolmitriptan in a solvent such as ethyl acetate and 2-propanol or by crystallizing from a solution of zolmitriptan in a solvent such as methanol and ethanol.

WO Patent publication No. 2006/055964 disclosed the solvated forms zolmitriptan Form B (dimethylformamide solvate), Form C (ethanol solvate), Form D (2-butanol or 1,3-dioxane solvate), Form E (1-butanol solvate), Form F (isobutanol solvate), Form G (tetrahydrofuran solvate), Form H (cyclohexanone solvate), Form I (1,4-dioxane solvate), Form J (piperidine solvate), Form K (solvate of Methanol or ethanol or dimethylformamide or acetonitrile), Form L (acetonitrile solvate), Form M (cyclopentanone solvate), Form N (methyl ethyl ketone), Form O (piperidine solvate), Form P (pyridine solvate), Form Q (diethylamine solvate), Form R (dichloromethane solvate), Form S (solvate of butyl acetate of n-butanol), Form T (ethyl acetate solvate) and amorphous form.

U.S. Patent Application No. 2006/0148868 disclosed two crystalline forms, form II, form III of zolmitriptan and also disclosed solid amorphous form of zolmitriptan.

WO Patent Publication No. 2008/081475 disclosed two crystalline forms, form I, form II of zolmitriptan and also disclosed process for the preparation of zolmitriptan form A.

One object of the present invention is to provide a novel isopropyl acetate solvate form of zolmitriptan and a process for preparing it.

According to another object of the present invention is to provide process for preparing zolmitriptan polymorph A.

### DETAILED DESCRIPTION OF THE INVENTION

In accordance with one aspect of the present invention, there is provided zolmitriptan isopropyl acetate solvate, characterized by peaks in the powder x-ray diffraction spectrum having 2θ angle positions at about 8.1, 11.4, 16.8, 17.5, 18.0, 19.5 and 21.6±0.2 degrees. The powdered x-ray diffractogram (PXRD) of zolmitriptan isopropyl acetate solvate is shown in FIG. 1.

In accordance with another aspect of the present invention, there is provided a process for preparing zolmitriptan isopropyl acetate solvate which comprises:

a) dissolving zolmitriptan in isopropyl acetate solvent; and
b) precipitating zolmitriptan isopropyl acetate solvate from the solution obtained in step (a).

Zolmitriptan used in the process of the present invention may be in the form of anhydrous, hydrated, non-solvated or solvated zolmitriptan. Thus, for example, zolmitriptan polymorph B, zolmitriptan monohydrate, zolmitriptan isopropanol solvate, zolmitriptan 1-butanol solvate, zolmitriptan anisole solvate, zolmitriptan ethyl methyl ketone solvate, zolmitriptan tetrahydrofuran solvate and zolmitriptan 1,4-dioxane solvate may be used. Preferably, zolmitriptan used is in the form of zolmitriptan isopropanol solvate.

In accordance with another aspect of the present invention, there is provided a process for the preparation of zolmitriptan polymorph A, which comprises slurrying zolmitriptan isopropyl acetate solvate with an organic solvent or a mixture of organic solvents.

The organic solvent is selected from the group consisting of isopropyl acetate, ethyl acetate, n-heptane, hexane, methyl tert-butyl ether, ethanol and diisopropyl ether. Preferable organic solvent is selected from isopropyl acetate and ethyl acetate.

Slurrying may be performed until the zolmitriptan is converted to the desired polymorph A.

In accordance with another aspect of the present invention, there is provided a process for the preparation of zolmitriptan polymorph A, which comprises heating zolmitriptan isopropyl acetate solvate at above 25 deg C.

Preferably heating may be performed at 25 deg C to 80 deg C. The heating may be carried out until zolmitriptan isopropyl acetate solvate is completely converted into polymorph A.

### BRIEF DESCRIPTION OF THE DRAWING

FIG. 1 is X-ray powder diffraction spectrum of zolmitriptan isopropyl acetate solvate.

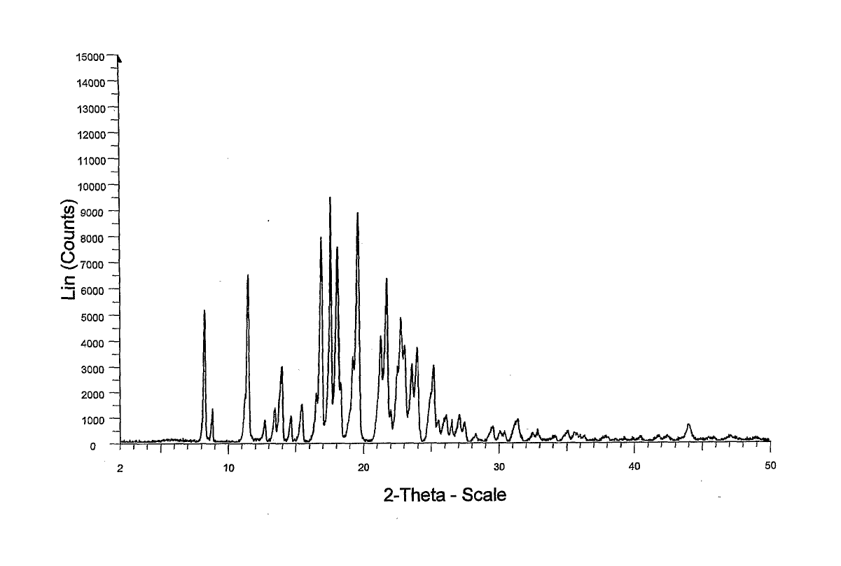

FIG. 2 is X-ray powder diffraction spectrum of zolmitriptan polymorph A.

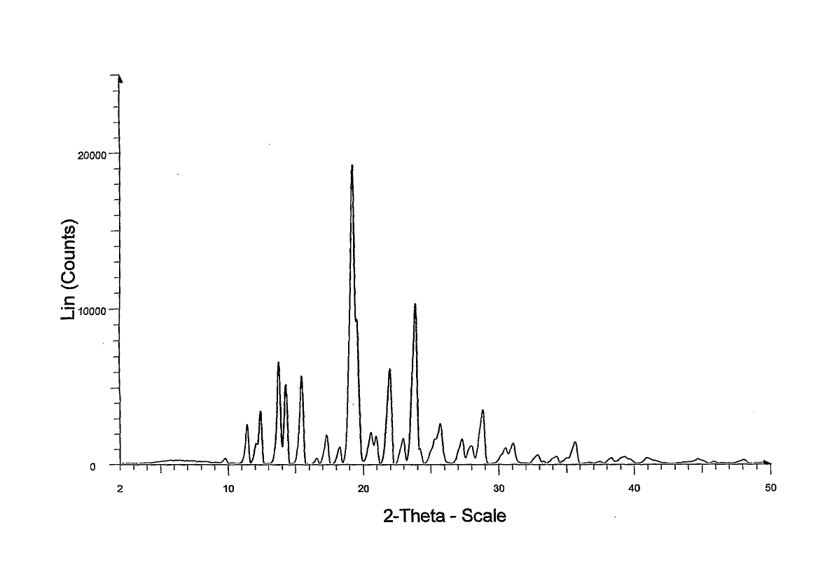

X-ray powder diffraction spectrum was measured on a bruker axs D8 advance X-ray powder diffractometer having a copper-Kα radiation. Approximately 1 gm of sample was gently flattered on a sample holder and scanned from 2 to 50 degrees two-theta, at 0.03 degrees to theta per step and a step of 0.5 seconds. The sample was simply placed on the sample holder. The sample was rotated at 30 rpm at a voltage 40 KV and current 35 mA.

The invention will now be further described by the following examples, which are illustrative rather than limiting.

### EXAMPLES

### Example 1

Sodium nitrite (16 gm) in water (120 ml) was added slowly for a period of 30 minutes at 0 deg C to a solution of (S)-4-(4-Aminobenzyl)-2-oxazolidinone (40 gm), concentrated hydrochloric acid (46 ml) and water (480 ml) in round bottomed flask, cooled to 0 deg C and stirred for 1 hour. The above diazotized solution was added for a period of 30 minutes at 0 deg C to sodium sulfite (78.3 gm) in water (200 ml) in another round bottomed flask, cooled to 0 deg C, slowly allowed to room temperature, heated to 55 deg C and stirred for 15 minutes at 60 deg C. Added concentrated hydrochloric acid (80 ml) to the reaction mass, stirred for 16 hours at 60 deg C, nitrogen gas was applied and heated to 90 deg C. Water (80 ml) was added to the reaction mass for 15 minutes at 90 deg C, added 4-(dimethylamino)-butyraldehyde diethylacetal for a period of 40 minutes, heated to reflux, stirred for 3 hours at reflux, cooled to 25-30 deg C and the pH was adjusted to 7 by adding sodium hydroxide solution (30%, 230 ml). Extracted with ethyl acetate (7×200 ml), adjusted the pH of the aqueous layer to 10 by adding sodium hydroxide solution (30%, 100 ml), heated to 50 deg C and again extracted with ethyl acetate (8×200 ml) at 50 deg C. Both the organic layers were combined, dried with sodium sulfate, given carbon treatment and the solvent was distilled off completely under vacuum at 50-55 deg C, ethyl acetate (80 ml) was added to the reaction mass at 25 deg C, stirred for 1 hour and cooled to 10 deg C. Stirred for 30 minutes at 10 deg C, filtered the material and washed with chilled ethylacetate (20 ml) under nitrogen atmosphere and dried at 45-50 deg C to yield 40 gm of (4S)-4-[[3-[2-(Dimethylamino)ethyl]-1H-indol-5-yl]methyl]-2-oxazolidinone.

(4S)-4-[[3-[2-(Dimethylamino)ethyl]-1H-indol-5-yl]methyl]-2-oxazolidinone, (40 gm, Zolmitriptan) was dissolved in isopropanol (200 ml) at 25 deg C, heated to reflux, stirred for 40 minutes at reflux and slowly allowed to cool to 0 deg C. Stirred the reaction mass for 1 hour at 0 deg C, filtered the compound, washed with chilled isopropanol(40 ml) and dried at 40-45 deg C under vacuum to yield (4S)-4-[[3-[2-(Dimethylamino)ethyl]-1H-indol-5-yl]methyl]-2-oxazolidinone isopropanol solvate (32 gm, Zolmitriptan isopropanol solvate; HPLC purity: 99.32%).

Zolmitriptan isopropanol solvate obtained above (32 gm) was dissolved in isopropyl acetate (2250 ml) at 25 deg C. Then the contents were heated to reflux and maintained for 30 minutes to form clear solution. The solution was cooled to 25 deg C during a period of 1 hour. The separated solid was filtered, washed with isopropyl acetate (160 ml) to obtain 32 gm of zolmitriptan isopropyl acetate solvate (HPLC purity: 99.8%).

### Example 2

To zolmitriptan isopropyl acetate solvate (5 gm) obtained as in example 1 was added isopropyl acetate (50 ml) at 25 deg C and slurried at reflux for 30 minutes. The contents were cooled to 25 deg C, stirred for 30 minutes at 25 deg C and filtered. The solid obtained was washed with chilled isopropyl acetate (7 ml), and then dried the solid at 60 deg C under vacuum to obtain 4.1 gm of zolmitriptan polymorph A (HPLC purity: 99.9%).

### Example 3

To zolmitriptan isopropyl acetate solvate (4 gm) was added ethyl acetate (40 ml) at 25 deg C and slurried at reflux for 30 minutes. The contents were cooled to 25 deg C for 30 minutes and filtered. The obtained solid was washed with ethyl acetate (5 ml) and dried at 60 deg C under vacuum to obtain 3.2 gm of zolmitriptan polymorph A.

### Example 4

Zolmitriptan isopropyl acetate solvate (4 gm) was heated 55 to 65 deg C for 5 hours under vacuum to obtain 3.1 gm of zolmitriptan polymorph A.

### Example 5

Zolmitriptan (5 gm) was dissolved in isopropyl alcohol (25 ml) at 25 deg C and heated to reflux, stirred for 30 minutes at reflux. The solution was slowly cooled to 0 deg C, stirred for 1 hour at 0 deg C and filtered. The solid obtained was washed with chilled isopropyl alcohol (5 ml) to obtain zolmitriptan isopropanol solvate (4 gm). The zolmitriptan isopropanol solvate obtained above was dissolved in isopropyl acetate (280 ml). The contents were heated to reflux and stirred for 30 minutes at reflux. The solution was slowly cooled to 25 deg C and stirred for 1 hour at 25 deg C. The separated solid was filtered, washed with isopropyl acetate (40 ml) to obtain 4 gm zolmitriptan isopropyl acetate solvate.

### Example 6

Zolmitriptan (4 gm) was dissolved in isopropyl acetate (280 ml) at 25 deg C. Then the contents were heated to reflux and maintained for 30 minutes to form clear solution. The solution was cooled to 25 deg C during a period of 1 hour. The separated solid was filtered, washed with isopropyl acetate (20 ml) to obtain 4 gm of zolmitriptan isopropyl acetate solvate.

We claim:

1. A zolmitriptan isopropyl acetate solvate, characterized by an X-ray powder diffractogram having peaks expressed as 2θ angle positions at about 8.1, 11.4, 16.8, 17.5, 18.0, 19.5 and 21.6±0.2 degrees.

2. A process for the preparation of zolmitriptan isopropyl acetate solvate as defined in claim 1; which comprises:
a. dissolving zolmitriptan in isopropyl acetate solvent; and
b. precipitating zolmitriptan isopropyl acetate solvate from the solution obtained in step (a).

3. The process as claimed in claim 2, wherein the zolmitriptan used from the form of anhydrous, hydrated, non-solvated or solvated zolmitriptan.

4. The process as claimed in claim 3, wherein the zolmitriptan used from the form of zolmitriptan polymorph B, zolmitriptan monohydrate, zolmitriptan isopropanol solvate, zolmitriptan 1-butanol solvate, zolmitriptan anisole solvate, zolmitriptan ethyl methyl ketone solvate, zolmitriptan tetrahydrofuran solvate and zolmitriptan 1,4-dioxane solvate.

5. The process as claimed in claim 4, wherein the zolmitriptan used is in the form of zolmitriptan isopropanol solvate.

6. A process for the preparation of the zolmitriptan polymorph A, which comprises slurrying zolmitriptan isopropyl acetate solvate with an organic solvent or a mixture of organic solvents.

7. The process as claimed in claim 6, wherein the organic solvent is selected from the group consisting of isopropyl acetate, ethyl acetate, n-heptane, hexane, methyl tert-butyl ether, ethanol and diisopropyl ether.

8. The process as claimed in claim 7, wherein the organic solvent is selected from isopropyl acetate and ethyl acetate.

9. A process for the preparation of the zolmitriptan polymorph A, which comprises heating zolmitriptan isopropyl acetate solvate at above 25 deg C.

10. The process as claimed in claim 9, wherein the heating is carried out at about 25 deg C to 80 deg C.
